(19) United States
(12) Patent Application Publication
(10) Pub. No.: US 2019/0233594 A1
(43) Pub. Date: Aug. 1, 2019

(54) SILICONE RESIN-LINEAR COPOLYMER AND RELATED METHODS

(71) Applicants: DOW SILICONES CORPORATION, Midland, MI (US); DOW CORNING TORAY CO., LTD., Chiyoda-ku, Tokyo (JP)

(72) Inventors: Haruhiko FURUKAWA, Ichihara-shi, Chiba (JP); John Bernard HORSTMAN, Midland, MI (US); Tomohiro IIMURA, Ichihara-shi, Chiba (JP); Tadashi OKAWA, Ichihara-shi, Chiba (JP); Steven SWIER, Midland, MI (US)

(73) Assignees: DOW SILICONES CORPORATION, Midland, MI (US); DOW CORNING TORAY CO., LTD., Chiyoda-ku , Tokyo (JP)

(21) Appl. No.: 16/339,291

(22) PCT Filed: Oct. 4, 2017
(86) PCT No.: PCT/US2017/055129

§ 371 Date: Apr. 3, 2019

Related U.S. Application Data
(60) Provisional application No. 62/403,952, filed on Oct. 4, 2016.

(51) Int. Cl. C08G 77/44; C08J 5/18; C09J 183/10; C09D 183/10; A61K 8/90

(52) U.S. Cl. CPC C08G 77/44; C08J 5/18; C08G 77/70; C09D 183/10; A61K 8/90; C09J 183/10

(57) ABSTRACT

A silicone resin-linear copolymer is disclosed which has a resinous structure (A1) including R¹SiO_{3/2} units, and a linear structure (A2) including repeated R²₂SiO_{2/2} units, wherein R¹ is a propyl group and each R² is an independently selected substituted or unsubstituted hydrocarbyl group, and wherein the resinous structure (A1) and the linear structure (A2) are bonded together in the silicone resin-linear copolymer via a siloxane bond. End use applications and related methods of the silicone resin-linear copolymer are also disclosed

### CROSS-REFERENCE TO RELATED APPLICATIONS

This application claims priority to and all advantages of U.S. Provisional Patent Application No. 62/403,952 filed on Oct. 4, 2016, the content of which is hereby incorporated by reference.

### FIELD OF THE INVENTION

The present invention generally relates to a resin-linear copolymer and, more specifically, to a silicone resin-linear copolymer, methods of its preparation, and various end use applications thereof.

### DESCRIPTION OF THE RELATED ART

Silicone resins typically include three-dimensional networks attributable to the presence of T siloxy units (R⁰SiO_{3/2}) and/or Q siloxy units (SiO_{4/2}), where R⁰ is a substituent. Properties of silicone resins differ depending on, among other things, their cross-link densities and molar fractions of siloxy units. Increasing the cross-link density generally results in a silicone resin having greater hardness and/or rigidity. For example, T resins typically form cage-like structures, and silica, or glass, consists of Q units.

Silicone polymers are typically linear or partially branched, and include repeating D siloxy units (R⁰₂SiO_{2/2}), typically capped with M siloxy units (R⁰₃SiO_{1/2}). Silicone polymers are often utilized to prepare silicone gels and elastomers, which are not as rigid or hard as silicone resins due to their lesser crosslink density and absence of three-dimensional networks.

### SUMMARY OF THE INVENTION

The present invention provides a silicone resin-linear co-polymer. The silicone resin-linear copolymer has a resinous structure (A1) including R¹SiO_{3/2} units, and a linear structure (A2) including repeated R²₂SiO_{2/2} units, wherein R¹ is a propyl group and each R² is an independently selected substituted or unsubstituted hydrocarbyl group, and wherein the resinous structure (A1) and the linear structure (A2) are bonded together in the silicone resin-linear copolymer via a siloxane bond.

The present invention additionally provides a composition. The composition comprises the (A) silicone resin-linear co-polymer; and (B) a carrier fluid.

Other end uses of the silicone resin-linear copolymer are also disclosed. For example, the silicone resin-linear polymer may be utilized as or with a film forming agent, as or with an adhesion promotor, as or with an encapsulant, etc.

The silicone resin-linear polymer also forms films having excellent physical properties. The present invention further provides an electronic device including a film formed from the silicone resin-linear co-polymer.

A method of forming a conformal coating on an electronic device is also provided. This method comprises applying the composition on the electronic device, and forming the conformal coating on the electronic device from the composition.

Methods of preparing the silicone resin-linear copolymer are also disclosed. In a first embodiment, the method comprises reacting a linear organopolysiloxane having at least one silicon-bonded hydroxyl group and an acetoxysilane to give an acetoxysilylated organopolysiloxane. In this first embodiment, the method further comprises reacting the acetoxysilylated organopolysiloxane with a silicone resin having at least one silicon-bonded hydroxyl group to give the silicone resin-linear copolymer.

In a second embodiment, the method comprises reacting a linear organopolysiloxane having at least one silicon-bonded hydrogen atom, hydroxyl group, or alkoxy group and a silicone resin having at least one silicon-bonded hydrogen atom, hydroxyl group, or alkoxy group in the presence of a Lewis acid catalyst. When the linear organopolysiloxane includes the silicon-bonded hydrogen atom, the silicone resin includes a silicon-bonded hydroxyl group or alkoxy group, and when the linear organopolysiloxane includes the silicon-bonded hydroxyl group or alkoxy group, the silicone resin includes the silicon-bonded hydrogen atom.

### DETAILED DESCRIPTION OF THE INVENTION

The present invention provides a silicone resin-linear copolymer. The silicone resin-linear copolymer has excellent properties and is well suited for diverse end use applications, including in compositions, to form films, etc. as described in greater detail below. Methods of preparing the silicone resin-linear copolymer are also disclosed.

Organopolysiloxanes contain siloxy units independently selected from [R₃SiO_{1/2}], [R₂SiO_{2/2}], [RSiO_{3/2}], or [SiO_{4/2}] siloxy units, where R may be, e.g., any organic group. These siloxy units are commonly referred to as M, D, T, and Q units, respectively. These siloxy units can be combined in various manners to form cyclic, linear, or branched structures. The chemical and physical properties of the resulting polymeric structures vary depending on the number and type of siloxy units in the organopolysiloxane. For example, “linear” organopolysiloxanes, or polymers, contain, in some embodiments, mostly D, or [R₂SiO_{2/2}] siloxy units, which results in polydiorganosiloxanes that are fluids of varying viscosities, depending on the “degree of polymerization” or “dp” as indicated by the number of D units in the polydiorganosiloxane. “Resinous” organopolysiloxanes, or silicone resins, include three dimensional structures attributable to siloxy units selected from T and/or Q siloxy units. Increasing the amount of T or Q siloxy units in an organopolysiloxane, in some embodiments, results in resins having increasing hardness and/or glass like properties.

As used herein “silicone resin-linear copolymers” refer to organopolysiloxanes containing “linear” D siloxy units in combination with “resin” T siloxy units. In particular, the silicone resin-linear copolymer has a resinous structure (A1) including R¹ SiO_{3/2} units, and a linear structure (A2) including repeated R²₂SiO_{2/2} units, wherein R¹ is a propyl group and each R² is an independently selected substituted or unsubstituted hydrocarbyl group.

Each R² is independently selected and may be the same as or different from one another. R² may independently be linear, branched, and/or cyclic. Cyclic hydrocarbyl groups encompass aryl groups as well as saturated or non-conjugated cyclic groups. Aryl groups may be monocyclic or polycyclic. Linear and branched hydrocarbyl groups may independently be saturated or unsaturated. For example, linear hydrocarbyl groups include alkyl groups, alkenyl groups, alkynyl groups, etc. One example of a combination of a linear and cyclic hydrocarbyl group is an aralkyl group. By “substituted,” it is meant that one or more hydrogen atoms may be replaced with atoms other than hydrogen (e.g. a halogen atom, such as chlorine, fluorine, bromine, etc.), or a carbon atom within the chain of R² may be replaced with an atom other than carbon, i.e., R² may include one or more heteroatoms within the chain, such as oxygen, sulfur, nitrogen, etc.

Typically, the hydrocarbyl groups of each R² independently comprise alkyl or aryl groups. Alkyl groups typically have from 1 to 30 carbon atoms, alternatively 1 to 24 carbon atoms, alternatively 1 to 20 carbon atoms, alternatively 1 to 12 carbon atoms, alternatively 1 to 10 carbon atoms, alternatively 1 to 6 carbon atoms, alternatively 1 to 4 carbon atoms, alternatively 1 to 3 carbon atoms, alternatively 1 or 2 carbon atoms, alternatively are methyl groups. Aryl groups typically have from 5 to 9 carbon atoms, alternatively 6 to 7 carbon atoms, and alternatively 5 to 6 carbon atoms.

The resinous structure (A1) and the linear structure (A2) are bonded together in the silicone resin-linear copolymer via a siloxane bond.

In some embodiments, the silicone resin-linear copolymer is a “block” copolymer, as opposed to “random” copolymer. The block copolymer refers to embodiments in which the linear structure (A2) is primarily bonded together to form at least one polymeric chain having, in some embodiments, an average of from 10 to 400 D units (e.g., an average of from 10 to 350 D units; 10 to 300 D units; 10 to 200 D units; 10 to 100 D units; 50 to 400 D units; 100 to 400 D units; 150 to 400 D units; 200 to 400 D units; 300 to 400 D units; 50 to 300 D units; 100 to 300 D units; 150 to 300 D units; 200 to 300 D units; 100 to 150 D units, 115 to 125 D units, 90 to 170 D units or 110 to 140 D units), which are referred herein as “linear blocks.” The number of D units may be referred to as the degree of polymerization, or DP. DP, as used herein, relates solely to the linear structure (A2) and not the resinous structure (A1). When the silicone resin-linear copolymer includes more than one linear structure (A2), the DP of each linear structure is independently selected.

The resinous structure (A1) generally comprises T units (i.e., [R¹SiO_{3/2}]) primarily bonded to each other to form three dimensional networks, or the resinous structure. The silicone resin-linear copolymer may comprise more than one resinous structure (A1), which, if present as a plurality, are typically bonded together via the linear structure (A2).

In some embodiments, the resinous structure (A1) has a number average molecular weight of at least 500 g/mole, e.g., at least 1000 g/mole, at least 2000 g/mole, at least 3000 g/mole or at least 4000 g/mole; or have a molecular weight of from 500 g/mole to 4000 g/mole, from 500 g/mole to 3000 g/mole, from 500 g/mole to 2000 g/mole, from 500 g/mole to 1000 g/mole, from 1000 g/mole to 2000 g/mole, from 1000 g/mole to 1500 g/mole, from 1000 g/mole to 1200 g/mole, from 1000 g/mole to 3000 g/mole, from 1000 g/mole to 2500 g/mole, from 1000 g/mole to 4000 g/mole, from 2000 g/mole to 3000 g/mole or from 2000 g/mole to 4000 g/mole. When the silicone resin-linear copolymer includes more than one resinous structure (A1), the number average molecular weight of each resinous structure is independently selected.

In certain embodiments, the silicone resin-linear copolymer consists essentially of, alternatively consists of, the resinous structure (A1) and the linear structure (A2). In these embodiments, the silicone resin-linear copolymer is substantially free, alternatively free, of M units and Q units.

In these embodiments, the resinous structure (A1) consists of R¹SiO_{3/2} units, wherein R¹ is defined above. Similarly, in these or other embodiments, the linear structure (A2) consists of R²₂SiO_{2/2} units, wherein R² is defined above.

As set forth above, the resinous structure (A1) and the linear structure (A2) are bonded together via a siloxane bond. This is distinguished from preparation via hydrosilylation, in which case a divalent organic alkylene group may be present between the resinous structure (A1) and the linear structure (A2). In certain embodiments, the siloxane bond is derived from an acetoxysilyl group. Alternatively, the siloxane bond may be derived from hydrolysis/condensation of any silicon-bonded hydrolysable group. Examples of hydrolysable groups when bonded to silicon include H, a halide group, an alkoxy group, an alkylamino group, a carboxy group, an alkyliminoxy group, an alkenyloxy, and an N-alkylamido group.

In other embodiments, the siloxane bond between the resinous structure (A1) and the linear structure (A2) is derived from dehydrogenation, and in particular dehydrogenation via a Lewis acid catalyst. Additional detail is provided below in regards to the method of preparing the silicone resin-linear polymer.

In certain embodiments, the silicone resin-linear copolymer has a weight average molecular weight (M_{w}) of at least 20,000 g/mole, alternatively a weight average molecular weight of at least 40,000 g/mole, alternatively a weight average molecular weight of at least 50,000 g/mole, alternatively a weight average molecular weight of at least 60,000 g/mole, alternatively a weight average molecular weight of at least 70,000 g/mole, or alternatively a weight average molecular weight of at least 80,000 g/mole. In some embodiments, the silicone resin-linear copolymer has a weight average molecular weight (M_{w}) of from about 20,000 g/mole to about 250,000 g/mole or from about 75,000 g/mole to about 120,000 g/mole, alternatively a weight average molecular weight of from about 40,000 g/mole to about 100,000 g/mole, alternatively a weight average molecular weight of from about 50,000 g/mole to about 100,000 g/mole, alternatively a weight average molecular weight of from about 50,000 g/mole to about 80,000 g/mole, alternatively a weight average molecular weight of from about 50,000 g/mole to about 70,000 g/mole, alternatively a weight average molecular weight of from about 50,000 g/mole to about 60,000 g/mole. In some embodiments, the silicone resin-linear copolymer has a number average molecular weight (Mₙ) of from about 80,000 to about 120,000 g/mole; or from about 90,000 to about 110,000 g/mole g/mole. The average molecular weight may be readily determined using Gel Permeation Chromatography (GPC) techniques based on polystyrene standards.

In the silicone resin-linear copolymer, the (A1) resinous structure includes silicon-bonded propyl groups, whereas the (A2) linear structure includes silicon-bonded hydrocarbyl groups, which are independently selected and may be substituted or unsubstituted. Typically, in the silicone resin-linear copolymer, the amount of silicon-bonded propyl groups constitute at least 10 mol %, alternatively at least 20 mol %, alternatively at least 30 mol %, based on all silicon-bonded organic groups.

In some embodiments, the silicone resin-linear copolymer contains residual silanol groups (—SiOH). The amount of silanol groups present in the silicone resin-linear copolymer may vary from 0.5 to 35 mole percent silanol groups (—SiOH),alternatively from 2 to 32 mole percent silanol groups (—SiOH), alternatively from 8 to 22 mole percent silanol groups (—SiOH).

The silanol groups may be present on any siloxy units within the silicone resin-linear copolymer, although such silanol groups are typically present in the (A1) resinous structure. For example, typically, the majority (e.g., greater than 75%, greater than 80%, greater than 90%) of the silanol groups will reside on the (A1) resinous structure. The presence of such silanol groups allows for further condensation or other reaction if desired. However, the presence of such silanol groups does not adversely impact shelf life of the silicone resin-linear copolymer.

The silicone resin-linear copolymer has many diverse end use applications. For example, the invention also provides a film-forming agent comprising the silicone resin-linear copolymer. The film-forming agent may comprise components in addition to the silicone resin-linear copolymer, including any components in conventional film-forming agents, e.g. carriers, etc.

The present invention also provides an adhesion promotor comprising the silicone resin-linear co-polymer. The adhesion promotor may comprise components in addition to the silicone resin-linear copolymer, including any components in conventional adhesion promotors, e.g. carriers, etc.

Further still, the present invention provides an encapsulant comprising the silicone resin-linear co-polymer. The encapsulant may further comprise a component reactive with the silicone resin-linear copolymer, or the silicone resin-linear copolymer itself may serve as the encapsulant upon application. The encapsulant may comprise components in addition to the silicone resin-linear copolymer, including any components in conventional encapsulants, e.g. carriers, etc. The encapsulant may be for, for example, electrical components, devices, photovoltaic cells, etc.

The present invention also provides a composition. The composition comprises the (A) silicone resin-linear copolymer; and (B) a carrier fluid. Suitable carrier fluids include silicones, both linear and cyclic, organic oils, organic solvents and mixtures of these. The carrier fluid may carry, alternatively partially solubilize, alternatively solubilize, the (A) silicone resin-linear copolymer.

In certain embodiments, the (B) carrier fluid comprises (B1) a volatile fluid at 25° C. Specific examples of suitable volatile fluids include a low viscosity silicone or a volatile methyl siloxane or a volatile ethyl siloxane or a volatile methyl ethyl siloxane having a viscosity at 25° C. in the range of from 1 to 1,000 mm²/sec. Specific examples thereof such as hexamethylcyclotrisiloxane, octamethylcyclotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsily)oxy)}trisiloxane, hexamethyl-3,3,bis{(trimethylsilyl)oxy}trisiloxane, pentamethyl{(trimethylsilyl)oxy}cyclotrisiloxane as well as polydimethylsiloxanes, polyethylsiloxanes, polymethylethylsiloxanes, polymethylphenylsiloxanes, polydiphenylsiloxanes.

Alternatively, the (B) carrier fluid may comprise an organic vehicle, exemplified by, but not limited to, aromatic hydrocarbons, aliphatic hydrocarbons, alcohols, aldehydes, ketones, amines, esters, ethers, glycols, glycol ethers, alkyl halides and aromatic halides. Hydrocarbons including isododecane, isohexadecane, Isopar L (C11-C13), Isopar H (C11-C12), hydrogentated polydecen. Ethers and esters including isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME). octyldodecyl neopentanoate, diisobutyl adipate, diisopropyl adipate, propylene glycol dicaprylate/dicaprate, and octyl palmitate. Additional organic carrier fluids suitable as a stand-alone compound or as an ingredient to the carrier fluid include fats, oils, fatty acids, and fatty alcohols.

The relative amounts of the (A) silicone resin-linear copolymer and the (B) carrier fluid may vary based on a desired end use application of the composition. In various embodiments, the composition comprises the (B) carrier fluid in an amount of from 0 to 98 weight percent, alternatively 0.5 to 90 weight percent, alternatively 5 to 80 weight percent, based on the total weight of the composition.

The composition has many end use applications. For example, the present invention provides a method of preparing a conformal coating on an electronic device (the “conformal coating method”). The conformal coating method comprises applying the composition on the electronic device. The conformal coating method further comprises forming the conformal coating on the electronic device.

The electronic device is not limited and may be referred to as a “microelectronic device” and/or an “electronic circuit.” Exemplary examples thereof include silicon based devices, gallium arsenide devices, focal plane arrays, opto-electronic devices, photovoltaic cells, optical devices, dielectric layers, doped dielectric layers to produce transistor-like devices, pigment loaded binder systems containing silicon to produce capacitors and capacitor-like devices, multilayer devices, 3-D devices, silicon-on-insulator (SOI) devices, super lattice devices and the like.

Applying the composition may comprise any suitable application technique. Typically, the composition is applied in wet form via a wet coating technique. In certain embodiments, the composition is applied by i) spin coating; ii) brush coating; iii) drop coating; iv) spray coating; v) dip coating; vi) roll coating; vii) flow coating; viii) slot coating; ix) gravure coating; or x) a combination of any of i) to ix). After applying the composition, the applied composition may be baked, dried, or annealed. The applied composition may be cured to give the conformal coating, or merely dried to give the conformal coating.

The conformal coating has a thickness that may vary depending upon its end use application. Typically, the film has a thickness of from greater than 0 to 100 micrometers (μm), alternatively from 10 to 90, alternatively 25 to 75, micrometers (μm). However, other thicknesses are contemplated, e.g. from 0.1 to 200 μm. For example, the thickness of the film may be from 0.2 to 175 μm; alternatively from 0.5 to 150 μm; alternatively from 0.75 to 100 μm; alternatively from 1 to 75 μm; alternatively from 2 to 60 μm; alternatively from 3 to 50 μm; alternatively from 4 to 40 μm; alternatively any one of 1 , 2, 3, 4, 5, 6, 7, 8, 9, 10, 15, 20, 25, 30, 35, 40, 45, 50, 60, 70, 75, 80, 90, 100, 150, 175, and 200 μm.

The present invention further provides an electronic device including a film formed from the silicone resin-linear copolymer. The film may be the conformal coating. However, the film may be something other than the conformal coating, and an interlayer or other portion of the electronic device. The film may be formed in accordance with any of the techniques disclosed above relative to the coating. The film may be dried, baked, cured, annealed, or otherwise processed. For example, the film may comprise the reaction product of the silicone resin-linear copolymer, or the film may comprise the silicone resin-linear copolymer itself.

In other embodiments, the composition may be a personal care composition, such as a cosmetic composition. In these embodiments, the composition further comprises (C) a personal care ingredient. For clarity and consistency, “the (C) personal care ingredient” encompasses embodiments where the composition includes but one or two or more personal care ingredients.

In certain embodiments, the (C) personal care ingredient comprises a skin care ingredient. In these embodiments, the composition may be referred to as a skin care composition. If utilized to prepare the composition, the skin care ingredient is typically selected from water phase stabilizing agents, cosmetic biocides, conditioning agents (which may be silicone, cationic, hydrophobic, etc.), emollients, moisturizers, colorants, dyes, ultraviolet (UV) absorbers, sunscreen agents, antiperspirants, antioxidants, fragrances, antimicrobial agents, antibacterial agents, antifungal agents, antiaging actives, anti-acne agents, skin-lightening agents, pigments, preservatives, pH controlling agents, electrolytes, chelating agents, plant extracts, botanical extracts, sebum absorbents, sebum control agents, vitamins, waxes, surfactants, detergents, emulsifiers, thickeners, propellant gases, skin protectants, film forming polymers, light-scattering agents and combinations thereof. With some of these skin care embodiments, the composition may be referred to as a sunscreen, a shower gel, a soap, a hydrogel, a cream, a lotion, a balm, foundation, lipstick, eyeliner, a cuticle coat, or blush. Various species of such skin care ingredients are known by one of ordinary skill in the art.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C₃₀₋₄₅ alkyl methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa-decane; branched C₈-C₁₆ esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid, and mixtures thereof.

Example of waxes include hydrocarbon waxes such as beeswax, lanolin wax, rice wax, carnauba wax, candelilla wax, microcrystalline waxes, paraffins, ozokerite, polyethylene waxes, synthetic wax, ceresin, lanolin, lanolin derivatives, cocoa butter, shellac wax, bran wax, capok wax, sugar cane wax, montan wax, whale wax, bayberry wax, silicone waxes (e.g. polymethylsiloxane alkyls, alkoxys and/or esters, C₃₀₋₄₅ alkyldimethylsilyl polypropylsilsesquioxane), and mixtures thereof.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives, and mixtures thereof.

Examples of surface active materials may be anionic, cationic or non ionic, and include organomodified silicones such as dimethicone copolyol; oxyethylenated and/or oxypropylenated ethers of glycerol; oxyethylenated and/or oxypropylenated ethers of fatty alcohols such as ceteareth-30, C₁₂₋₁₅ pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives; and mixtures thereof.

Further examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, and mixtures thereof.

Nonionic surfactants include dimethicone copolyols, fatty acid esters of polyols, for instance sorbitol or glyceryl mono-, di-, tri- or sesqui-oleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Anionic surfactants include carboxylates (sodium 2-(2-hydroxyalkyloxy)acetate)), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

Examples of thickeners include acrylamide copolymers, acrylate copolymers and salts thereof (such as sodium polyacrylate), xanthan gum and derivatives, cellulose gum and cellulose derivatives (such as methylcellulose, methylhydroxypropylcellulose, hydroxypropylcellulose, polypropylhydroxyethylcellulose), starch and starch derivatives (such as hydroxyethylamylose and starch amylase), polyoxyethylene, carbomer, sodium alginate, arabic gum, cassia gum, guar gum and guar gum derivatives, cocamide derivatives, alkyl alcohols, gelatin, PEG-derivatives, saccharides (such as fructose, glucose) and saccharides derivatives (such as PEG-120 methyl glucose diolate), and mixtures thereof.

Examples of water phase stabilizing agents include electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, ciclopirox olamine, hexamidine, oxyquinoline and its derivatives, PVP-iodine, zinc salts and derivatives such as zinc pyrithione, and mixtures thereof.

Examples of sebum absorbants or sebum control agents include silica silylate, silica dimethyl silylate, dimethicone/vinyl dimethicone crosspolymer, polymethyl methacrylate, cross-linked methylmethacrylate, aluminum starch octenylsuccinate, and mixtures thereof.

Examples of pigments and colorants include surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include aromatic types including azo, indigoid, triphenylmethane, anthraquinone, and xanthine dyes which are designated as D&C and FD&C blues, browns, greens, oranges, reds, yellows, etc, and mixtures thereof. Surface treatments include those treatments based on lecithin, silicone, silanes, fluoro compounds, and mixtures thereof.

Examples of silicone conditioning agents include silicone oils such as dimethicone; silicone gums such as dimethiconol; silicone resins such as trimethylsiloxy silicate, polypropyl silsesquioxane; silicone elastomers; alkylmethylsiloxanes; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16; saccharide functional siloxanes; carbinol functional siloxanes; silicone polyethers; siloxane copolymers (divinyldimethicone/dimethicone copolymer); acrylate or acrylic functional siloxanes; and mixtures or emulsions thereof

Examples of cationic conditioning agents include guar derivatives such as hydroxypropyltrimethylammonium derivative of guar gum; cationic cellulose derivatives, cationic starch derivatives; quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polymeric quaternary ammonium salts of hydroxyethyl cellulose reacted with a fatty alkyl dimethyl ammonium substituted epoxide; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats, e.g. Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, stearyltrimethylammonium chloride, and mixtures thereof. In some instances, the cationic conditioning agent is also hydrophobically modified, such as hydrophobically modified quaternized hydroxyethylcellulose polymers; cationic hydrophobically modified galactomannan ether; and mixtures thereof.

Examples of hydrophobic conditioning agents include guar derivatives; galactomannan gum derivatives; cellulose derivatives; and mixtures thereof.

UV absorbers and sunscreen agents include those which absorb ultraviolet light between 290-320 nanometers (the UV-B region) and those which absorb ultraviolet light in the range of 320-400 nanometers (the UV-A region). UV absorbers and sunscreen agents also include those which absorb infrared light in the infrared spectrum (700 nanometers to 1 millimeter).

Some examples of sunscreen agents are aminobenzoic acid, cinoxate, diethanolamine methoxycinnamate, digalloyl trioleate, dioxybenzone, ethyl 4-[bis(Hydroxypropyl)] aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, trolamine salicylate, and mixtures thereof

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, octyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, triPABA panthenol, urocanic acid, VA/crotonates/methacryloxybenzophenone-1 copolymer, and mixtures thereof.

Examples of antiperspirant agents and deodorant agents include aluminum chloride, aluminum zirconium tetrachlorohydrex GLY, aluminum zirconium tetrachlorohydrex PEG, aluminum chlorohydrex, aluminum zirconium tetrachlorohydrex PG, aluminum chlorohydrex PEG, aluminum zirconium trichlorohydrate, aluminum chlorohydrex PG, aluminum zirconium trichlorohydrex GLY, hexachlorophene, benzalkonium chloride, aluminum sesquichlorohydrate, sodium bicarbonate, aluminum sesquichlorohydrex PEG, chlorophyllin-copper complex, triclosan, aluminum zirconium octachlorohydrate, zinc ricinoleate, and mixtures thereof.

Examples of skin protectants include allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, and mixtures thereof.

Examples of dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1 H-pyrazole-sulfate; 2,3-diaminodihyd ropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C₂₁-C₂₂ isoalkyl acidate; isatin; Isatis tinctoria leaf powder; 2-methoxymethyl-p-phenylenediamine sulfate; 2-methoxy-p-phenylenediamine sulfate; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o- phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene.

Examples of fragrances include perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, Gamma-Methyl so-called Ionone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)ppropyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexylon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran.

In specific embodiments, the perfume ketones are selected for odor character from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

In specific embodiments, the perfume aldehyde is selected for odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carbox aldehyde, 5 or 6 methoxy 10 hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl-1-cyclohexen-1-yl)-2-butena I, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, zinc dibutyldithiocarbamate, and mixtures thereof.

Examples of propellant gases include carbon dioxide, nitrogen, nitrous oxide, volatile hydrocarbons such as butane, isobutane, or propane, and chlorinated or fluorinated hydrocarbons such as dichlorodifluoromethane and dichlorotetrafluoroethane or dimethylether; and mixtures thereof.

In a specific embodiment, the composition is a sunscreen. In these embodiments, personal care ingredient comprises the sunscreen agent. The sunscreen agent may be, for example, a sunscreen additive, an SPF booster, a photostabilizer, a film-forming polymer, etc. The sunscreen may be also or alternatively be utilized in sunless tanning applications. Specific examples of sunscreen agents are set forth above.

In other embodiments, the (C) personal care ingredient comprises a hair care ingredient. In these embodiments, the composition may be referred to as a hair care composition. If utilized to prepare the composition, the hair care ingredient is typically selected from conditioning agents (which may be silicone, cationic, hydrophobic, etc.), colorants, dyes, ultraviolet (UV) absorbers, preservatives, plant extracts, fatty alcohols, vitamins, fragrance, anti-dandruff agents, color care additives, pearlising agents, pH controlling agents, electrolytes, chelating agents, styling agents, ceramides, amino-acid derivatives, suspending agents, surfactants, detergents, emulsifiers, thickeners, oxidizing agents, reducing agents, film-forming polymers, and combinations thereof. With some of these hair care embodiments, the composition may be referred to as a shampoo, a rinse-off conditioner, a leave-in conditioner, a gel, a pomade, a serum, a spray, a coloring product, or mascara. Examples of many of these hair care ingredients are set forth above as suitable personal care ingredients.

Examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof.

Examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Examples of antidandruff agents include pyridinethione salts, selenium compounds such as selenium disulfide, and soluble antidandruff agents, and mixtures thereof.

In other embodiments, the (C) personal care ingredient comprises a nail care ingredient. In these embodiments, the composition may be referred to as a nail care composition. If utilized to prepare the composition, the nail care ingredient may be any ingredient utilized in nail care compositions, e.g. nail polishes, nail gels, nail tips, acrylic finishes, etc. Examples of such nail care ingredients include pigments, resins, solvents, volatile halogenated compounds (e.g. methoxynonafluorobutane and/or ethoxynonafluorobutane), etc.

More specifically, examples of nail care ingredients include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder; and mixtures thereof.

In other embodiments, the (C) personal care ingredient comprises a tooth care ingredient. In these embodiments, the composition may be referred to as a tooth care composition. One specific example of such a tooth care composition is toothpaste. Another example of a tooth care composition is a tooth whitening composition. The tooth care ingredient may be any tooth care ingredient suitable for the tooth care composition, such as an abrasive compound (e.g. aluminum hydroxide, calcium carbonate, silica, zeolite), a fluoride compound, a surfactant, a flavorant, a remineralizer, an antibacterial agent, etc.

In certain embodiments, the (C) personal care ingredient comprises a film-forming polymer, which may be utilized as the (C) personal care ingredient whether the composition is utilized for skin care, hair care, etc. “Film-forming polymer,” as used herein, means a polymer or oligomer which is capable of, by itself or optionally in the presence of a film-forming agent, forming a film on a substrate. The film-forming polymer may form the film upon an application of a curing condition, e.g. the application of heat, exposure to atmospheric conditions, etc. Alternatively, the film-forming polymer may form the film upon evaporation of any carrier vehicle in which the film-forming polymer may optionally be disposed. The film-forming polymer may undergo a reaction, e.g. the film-forming polymer may become cross-linked or otherwise include additional bonds, when forming the film. However, the film-forming polymer may form the film in the absence of such a reaction. The film-forming polymer may be a gelling agent. The film-forming polymer is particularly advantageous when the composition is the sunscreen, although the (C) personal care ingredient may comprise the film-forming polymer in other compositions as well.

The substrate on which the film is formed may be any substrate, although the substrate is generally a portion of a mammal, particularly a human, as described in greater detail below with reference to the treatment method. Specific examples of suitable substrates include skin, hair, and nails.

Generally, the film is continuous, although the film may have a varying thickness. By continuous, it is meant that the film does not define any apertures. The film may be referred to as being macroscopically continuous. The film may be supported by the substrate, or may be bonded, e.g. physically and/or chemically, to the substrate. In certain embodiments, the film is optionally removable from the substrate, e.g. the film may be peelable from the substrate. The film may remain intact as a free-standing film upon being separated from the substrate or may be separated through application of shear, which may damage and/or destroy continuity of the film.

Specific examples of film-forming polymers that are suitable include acrylic polymers, polyurethanes, polyurethane-acrylics, polyesters, polyester-polyurethanes, polyether-polyurethanes, polyesteramides, alkyds, polyamides, polyureas, polyurea-polyurethanes, cellulose-based polymers (e.g. nitrocellulose), silicones, acrylic-silicones, polyacrylamides, fluoropolymers, polyisoprenes, and any copolymers or terpolymers thereof or including one of these. The term “silicones,” as used herein with reference to suitable film-forming polymers, includes linear, branched, and resinous silicones, although resinous silicones are generally referred to as silicone resins rather than polymers. The silicone may be modified, e.g. the silicone may be a silicone-grafted acrylic polymer.

As introduced above, the film-forming polymer may be disposed in a carrier vehicle, which may partially or fully solubilize the film-forming polymer. Depending on a selection of the film-forming polymer, the carrier vehicle may be, for example, an oil, e.g. an organic oil and/or a silicone oil, a solvent, water, etc. The film-forming polymer may be in the form of polymer particles, which are optionally surface-stabilized with at least one stabilizer, and the polymer particles may be present as a dispersion or emulsion.

The film-forming polymer may be a block polymer, which may be styrene-free. Typically, the block polymer comprises at least one first block and at least one second block, which may be linked together via an intermediate block comprising at least one constituent monomer of the first block and at least one constituent monomer of the second block. Generally, the glass transition temperatures of the first and second blocks are different from one another.

Monomers that may be utilized to prepare the block polymer include, for example, methyl methacrylate, isobutyl (meth)acrylate and isobornyl (meth)acrylate, methyl acrylate, isobutyl acrylate, n-butyl methacrylate, cyclodecyl acrylate, neopentyl acrylate, isodecylacrylamide 2-ethylhexyl acrylate and mixtures thereof.

In specific embodiments, the film-forming polymer be obtained or generated via free-radical polymerization. For example, the film-forming polymer may be generated via free-radical polymerization of at least one acrylic monomer and at least one silicone- or hydrocarbon-based macromonomer including a polymerizable end group.

Specific examples of hydrocarbon-based macromonomers include homopolymers and copolymers of linear or branched C₈-C₂₂ alkyl acrylate or methacrylate. The polymerizable end group may be a vinyl group or a (meth)acrylate group, e.g. poly(2-ethylhexyl acrylate) macromonomers; poly(dodecyl acrylate) or poly(dodecyl methacrylate) macromonomers; poly(stearyl acrylate) or poly(stearyl methacrylate) macromonomers, etc. Such macromonomers generally include one (meth)acrylate group as the polymerizable end group.

Additional examples of hydrocarbon-based macromonomers include polyolefins containing an ethylenically unsaturated end group (as the polymerizable end group), e.g. a (meth)acrylate end group. Specific examples of such polyolefins include polyethylene macromonomers, polypropylene macromonomers, polyethylene/polypropylene copolymer macromonomers, polyethylene/polybutylene copolymer macromonomers, polyisobutylene macromonomers; polybutadiene macromonomers; polyisoprene macromonomers; polybutadiene macromonomers; and poly (ethylene/butylene)-polyisoprene macromonomers.

Examples of silicone-based macromonomers include organopolysiloxanes containing the polymerizable end group, e.g. a (meth)acrylate end group. The organopolysiloxane may be linear, branched, partially branched, or resinous. In various embodiments, the organopolysiloxane is linear. In these embodiments, the organopolysiloxane may be polydimethylsiloxane, although hydrocarbon groups other than methyl groups may be present therein along with or in lieu of methyl groups. Typically, the polymerizable end group is terminal, although the polymerizable end group may optionally be pendant. One specific example of a silicone-based macromonomer is a monomethacryloxypropyl polydimethylsiloxane.

In certain embodiments, the film-forming polymer is an organic film-forming polymer that is soluble in oil as the carrier vehicle. In these embodiments, the film-forming polymer may be referred to as a liposoluble polymer. The liposoluble polymer may be of any type and specific examples thereof include those comprising or formed from olefins, cycloolefins, butadiene, isoprene, styrene, vinyl ethers, vinyl esters, vinyl amides, (meth)acrylic acid esters or amides, etc.

In one embodiment, the lipsoluble polymer is formed from monomers selected from the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, and combinations thereof.

Alternatively still, the lipsoluble polymer may be an acrylic-silicone grafted polymer, which typically includes a silicone backbone and acrylic grafts or alternatively includes an acrylic backbone and silicone grafts.

The film-forming polymer may be halogenated, e.g. the film-forming polymer may include fluorine atoms.

Alternatively as introduced above, the film-forming polymer may be a cellulose-based polymer, such as nitrocellulose, cellulose acetate, cellulose acetobutyrate, cellulose acetopropionate or ethylcellulose. Alternatively still, the film-forming polymer may comprise a polyurethane, an acrylic polymer, a vinyl polymer, a polyvinyl butyral, an alkyd resin, or resins derived from aldehyde condensation products, such as arylsulfonamide-formaldehyde resins.

Further, as introduced above, the film-forming polymer may comprise the silicone, which may be linear, branched, or resinous. Resinous silicones generally include at least one T and/or Q unit, as understood in the art. Examples of resinous silicones include silsesquioxanes. The silicone may include any combination of M, D, T, and Q units so long as the silicone constitutes the film-forming polymer.

When the film-forming polymer comprises the silicone, the film-forming polymer may comprise an amphiphilic silicone. Amphiphilic silicones typically contain a silicone portion which is compatible with a silicone medium, and a hydrophilic portion. The hydrophilic portion may be, for example, the residue of a compound selected from alcohols and polyols, having 1 to 12 hydroxyl groups, and polyoxyalkylenes (e.g. those containing oxypropylene units and/or oxyethylene units).

The amphiphilic silicone may be an oil with or without gelling activity. Oils of this kind may comprise, for example, dimethicone copolyols.

In one embodiment, the film-forming polymer comprises a silicone organic elastomer gel. Silicone organic elastomer gels comprise linear organopolysiloxane chains crosslinked via polyoxyalkylenes. The silicone organic elastomer gel may further include hydrophilic polyether functionality pending from the linear organopolysiloxane chains. Specific examples of suitable silicone organic elastomer gels are disclosed in International (PCT) Appln. No. PCT/US2010/020110, which is incorporated by reference herein in its entirety.

Additional examples of cross-linked silicone compounds suitable for use as the film-forming polymer are disclosed in U.S. application Ser. Nos. 10/269,758 and 10/228,890, the contents of which are incorporated by reference herein in their respective entireties. Additional examples of other film-forming polymers suitable for the composition are disclosed in International (PCT) Serial Nos. PCT/EP2007/064259, PCT/EP2007/060682, and PCT/EP2005/013018, which are each incorporated by reference herein in their respective entireties.

When the (C) personal care ingredient comprises the film-forming polymer, the film-forming polymer may be present in the composition in various amounts, e.g. from greater than 0 to less than 100, alternatively from 0.1 to 60, alternatively from 0.1 to 50 percent by weight based on the total weight of the composition. Combinations of different types of film-forming polymers may be utilized.

In various embodiments, the (C) personal care ingredient may comprise or be referred to as a personal care active, a health care active, or combination thereof (collectively “active” or “actives”). As used herein, a “personal care active” means any compound or mixtures of compounds that are known in the art as additives in personal care formulations, typically for providing a cosmetic and/or aesthetic benefit. A “healthcare active” means any compound or mixtures of compounds that are known in the art to provide a pharmaceutical or medical benefit. Thus, “healthcare active” includes materials considered as an active ingredient or active drug ingredient as generally used and defined by the United States Department of Health & Human Services Food and Drug Administration, contained in Title 21, Chapter I, of the Code of Federal Regulations, Parts 200-299 and Parts 300-499. Specific personal care actives and health care actives are described below. These personal care actives and health care actives may constitute the (C) personal care ingredient whether the (C) personal care ingredient is utilized to form, for example, the skin care composition, the hair care composition, the nail care composition, and/or the tooth care composition. For example, in various embodiments, the same personal care ingredient may be utilized to form either the hair care composition or the skin care composition. As understood in the art, at least some of the personal care actives described below are species of certain personal care ingredients introduced above with respect to the skin care composition, the hair care composition, the nail care composition, and the tooth care composition, respectively. For example, numerous species of plant or vegetable extracts are described below, which are exemplary examples of plant extracts set forth above as suitable personal care ingredients. The active ingredients or actives described below may constitute the (C) personal care ingredient of the composition or may be utilized in combination therewith.

Useful active ingredients for use in the composition include vitamins and vitamin derivatives, including “pro-vitamins”. Vitamins useful herein include, but are not limited to, Vitamin A1, retinol, C2-C18 esters of retinol, vitamin E, tocopherol, esters of vitamin E, and mixtures thereof. Retinol includes trans-retinol, 1, 3-cis-retinol, 11-cis-retinol, 9-cis-retinol, and 3,4-didehydro-retinol, Vitamin C and its derivatives, Vitamin B1, Vitamin B2, Pro Vitamin B5, panthenol, Vitamin B6, Vitamin B12, niacin, folic acid, biotin, and pantothenic acid. Other suitable vitamins and the INCI names for the vitamins considered included herein are ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate. In general, retinol, all trans retinoic acid and derivatives, isomers and analogs thereof, are collectively termed “retinoids”.

RETINOL, it should be noted, is an International Nomenclature Cosmetic Ingredient Name (INCI) designated by The Cosmetic, Toiletry, and Fragrance Association (CTFA), Washington DC, for vitamin A. Other suitable vitamins and the INCI names for the vitamins considered included herein are RETINYL ACETATE, RETINYL PALMITATE, RETINYL PROPIONATE, α-TOCOPHEROL, TOCOPHERSOLAN, TOCOPHERYL ACETATE, TOCOPHERYL LINOLEATE, TOCOPHERYL NICOTINATE, and TOCOPHERYL SUCCINATE.

Some examples of commercially available products suitable for use herein are Vitamin A Acetate and Vitamin C, both products of Fluka Chemie AG, Buchs, Switzerland; COVI-OX T-50, a vitamin E product of Henkel Corporation, La Grange, Ill.; COVI-OX T-70, another vitamin E product of Henkel Corporation, La Grange, Ill.; and vitamin E Acetate, a product of Roche Vitamins & Fine Chemicals, Nutley, N.J.

The active can be a protein, such as an enzyme. Enzymes include, but are not limited to, commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include, but are not limited to, proteases (bacterial, fungal, acid, neutral or alkaline), amylases (alpha or beta), lipases, mannanases, cellulases, collagenases, lisozymes, superoxide dismutase, catalase, and mixtures thereof. Protease include, but are not limited to, trypsin, chymotrypsin, pepsin, pancreatin and other mammalian enzymes; papain, bromelain and other botanical enzymes; subtilisin, epidermin, nisin, naringinase(L-rhammnosidase) urokinase and other bacterial enzymes. Lipase include, but are not limited to, triacyl-glycerol lipases, monoacyl-glycerol lipases, lipoprotein lipases, e.g. steapsin, erepsin, pepsin, other mammalian, botanical, bacterial lipases and purified ones. In a specific embodiment, natural papain is utilized as the enzyme. Further, stimulating hormones, e.g. insulin, can be used together with the enzyme(s) to boost effectiveness.

The active may also be one or more plant or vegetable extract. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix (Ivy) extract, hawthorn extract, Sambucus nigra extract, Achillea millefolium extract, Mentha piperita extract, sage extract, mallow extract, Cnidium officinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extract, Nelumbo nucifera extract, parsley extract, honey, hamamelis extract, Parietaria extract, Isodonis herba extract, bisabolol extract, Loquat extract, coltsfoot extract, butterbur extract, Pond cocos wolf extract, extract of butcher's broom, grape extract, propolis extract, luffa extract, safflower extract, peppermint extract, linden tree extract, Paeonia extract, hop extract, pine tree extract, horse chestnut extract, Mizu-bashou [Lysichiton camtschatcese] extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, royal jelly extract, and combinations thereof.

Representative, non-limiting examples of healthcare actives useful as drugs in the present compositions are described below. One or more of the drugs can be used, either alone or in combination with the actives and/or personal care ingredients described above.

The composition may include an antiparasite agent. The antiparasite agent can be of any type. Examples of antiparasite agents include, but are not limited to, hexachlorobenzene, carbamate, naturally occurring pyrethroids, permethrin, allethrin, malathion, piperonyl butoxide, and combinations thereof.

The composition may include an antimicrobial agent, also referred to as germicidal agent. The antimicrobial agent can be of any type. Examples of antimicrobial agents include, but are not limited to, phenols, including cresols and resorcinols. Such compositions may be used to treat infections of the skin. An example of a very common skin infection is acne, which involve infestation of the sebaceous gland with p. acnes, as well as Staphylococus aurus or Pseudomonas. Examples of useful antiacne actives include the keratolytics such as salicylic acid (o-hydroxybenzoic acid), derivatives of salicylic acid such as 5-octanoyl salicylic acid, and resorcinol; retinoids such as retinoic acid and its derivatives (e.g. cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate and cholate; parachlorometaxylenol; and combinations thereof.

Phenols, in concentrations of 0.2, 1.0, and 1.3, % by weight, are generally bacteriostatic, bactericidal, and fungicidal, respectively. Several phenol derivatives are more potent than phenol itself, and the most important among these are the halogenated phenols and bis-phenols, the alkyl-substituted phenols and the resorcinols. Hydrophobic antibacterials include triclosan, triclocarbon, eucalyptol, menthol, methylsalicylate, thymol, and combinations thereof.

The composition may include an antifungal agent. The antifungal agent can be of any type. Examples of antifungal agents include, but are not limited to, azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and combinations thereof. U.S. Pat. No. 4,352,808 discloses 3-aralkyloxy-2, 3-dihydro-2-(1H-imidazolylmethyl)benzo[b]thiophene compounds having antifungal and antibacterial activity, which is incorporated herein by reference.

The composition may include a steroidal anti-inflammatory agent. The steroidal anti-inflammatory agent can be of any type. Examples of steroidal anti-inflammatory agents include, but are not limited to, corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and combinations thereof.

Topical antihistaminic preparations currently available include 1 percent and 2 percent diphenhydramine (Benadryl® and Caladryl®), 5 percent doxepin (Zonalon®) cream, phrilamine maleate, chlorpheniramine and tripelennamine, phenothiazines, promethazine hydrochloride (Phenergan®) and dimethindene maleate. These drugs, as well as additional antihistamines can also be included in the composition. Additionally, so-called “natural” anti-inflammatory agents may be useful. For example, candelilla wax, alpha bisabolol, aloe vera, Manjistha (extracted from plants in the genus Rubia, particularly Rubia cordifolia), and Guggal (extracted from plants in the genus Commiphora, particularly Commiphora mukul, may be used as an active in the composition.

The composition may include a non-steroidal anti-inflammatory drug (NSAID). The NSAID can be of any type. Examples of NSAIDs include, but are not limited to, the following NSAID categories: propionic to acid derivatives; acetic acid derivatives; fenamic acid derivatives; biphenylcarboxylic acid derivatives; and oxicams. Such NSAIDs are described in the U.S. Pat. No. 4,985,459 which is incorporated herein by reference. Further examples include, but are not limited to, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, mniroprofen, tioxaprofen, suprofen, alminoprofen, tiaprofenic acid, fluprofen, bucloxic acid, and combinations thereof.

The composition may include an antioxidant/radical scavenger. The antioxidant can be of any type. Examples of antioxidants include, but are not limited to, ascorbic acid (vitamin C) and its salts, tocopherol (vitamin E), and its derivatives such as tocopherol sorbate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, the ascorbyl esters of fatty acids, amines (e.g. N,N-diethylhydroxylamine, amino-guanidine), sulfhydryl compounds (e.g. glutathione), and dihydroxy fumaric acid and its salts may be used, as well as EDTA, BHT and the like, and combinations thereof.

The composition may include an antibiotic. The antibiotic can be of any type. Examples of antibiotics include, but are not limited to, chloramphenicol, tetracyclines, synthetic and semi-synthesic penicillins, beta-lactames, quinolones, fluoroquinolnes, macrolide antibiotics, peptide antibiotics, cyclosporines, erythromycin, clindamycin, and combinations thereof.

The composition may include a topical anesthetic. The topical anesthetic can be of any type. Examples of topical anesthetics include, but are not limited to, benzocaine, lidocaine, bupivacaine, chlorprocaine, dibucaine, etidocaine, mepivacaine, tetracaine, dyclonine, hexylcaine, procaine, cocaine, ketamine, pramoxine, phenol, pharmaceutically acceptable salts thereof, and combinations thereof.

The composition may include an anti-viral agent. The anti-viral agent can be of any type. Examples of anti-viral agents include, but are not limited to, proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. In particular, anti-viral agents include, but are not limited to, nucleoside analogs (e.g. zidovudine, acyclovir, acyclovir prodrugs, famciclovir, gangcyclovir, vidarabine, idoxuridine, trifluridine, and ribavirin), n-docosanoll foscarnet, amantadine, rimantadine, saquinavir, indinavir, ritonavir, idoxuridine alpha-interferons and other interferons, AZT, and combinations thereof.

The composition may include an anti-cancer drug. The anti-cancer drug can be of any type. Examples of anti-cancer drugs include, but are not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin-2 (including recombinant interleukin 2, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; and combinations thereof.

Other anti-cancer drugs include, but are not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epothilone A; epothilone B; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; U.S. Pat. No. 6,162,432, which is incorporated herein by reference); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MWF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl-lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; pennyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; zinostatin stimalamer; and combinations thereof.

Additional examples of actives include analgesic agents and antihypertensive agents. Analgesic agents are known in the art and are colloquially referred to as painkillers. The analgesic agent may be selected from any known analgesic agents, and specific examples thereof include paracetamol (acetaminophen), morphine, codeine, heroine, methadone, thebaine, orpiarine, buprenorphine, morphinans, benzomorphans, acetaminophen, butorphanol, diflunisal, fenoprofen, fentanyl, fentanyl citrate, hydrocodone, aspirin, sodium salicylate, ibuprofen, oxymorphone, pentaxicine, naproxen, nalbuphine, mefenamic acid, meperidine and dihydroergotamine, non-steroidal anti-inflammatory agents, such as salicylates, and opioid agents, such as morphine and oxycodone. Antihypertensive agents are known in the art for treating or reducing hypertension, i.e., high blood pressure. The antihypertensive agent may be selected from any known antihypertensive agents, and specific examples thereof include diuretics, adrenergic receptor antagonists (e.g. beta blockers), benzodiazepines, calcium channel blockers, renin inhibitors, etc.

A typical narcotic antagonist is haloxone. Exemplary antitussive agents include, without limitation, diphenhydramine, guaifenesin, hydromorphone, ephedrine, phenylpropanolamine, theophylline, codeine, noscapine, levopropoxyphene, carbetapentane, chlorpehndianol and benzonatate.

Among the sedatives which may be utilized are, without limitation, chloral hydrate, butabarbital, alprazolam, amobarbital, chlordiazepoxide, diazepam, mephobarbital, secobarbital, diphenhydramine, ethinamate, flurazepam, halazepam, haloperidol, prochlorperazine, oxazepam, and talbutal.

Examples of cardiac drugs are, without limitation, quinidine, propranolol, nifedipine, procaine, dobutamine, digitoxin, phenyloin, sodium nitroprusside, nitroglycerin, verapamil HCl, digoxin, nicardipine HCl, and isosorbide dinitrate.

Antiemetics are illustrated by, without limitation, thiethylperazine, metoclopramide, cyclizine, meclizine, prochlorperazine, doxylamine succinate, promethazine, triflupromazine, and hydroxyzine.

A typical dopamine receptor agonist is bromocriptine mesylate. Exemplary amino acid, peptide and protein hormones include, without limitation, thyroxine, growth hormone (GH), interstitial cell stimulating hormone (ICSH), follicle-stimulating hormone (FSH), thyrotropic hormone (TSH), adrenocorticotropic hormone (ACTH), gonadotropin releasing hormone (GnRH) such as leuprolide acetate, vasopressin and their active degradation products Some products may have sufficiently high molecular weights that absorption through the stratum corneum or mucous membranes may be difficult. Therefore, the invention is applicable only to those hormones which have molecular weights and stereo configurations which will allow passage through the skin.

Female sex hormones which can be used include, without limitations, estradiol, diethylstilbestrol, conjugated estrogens, estrone, norethindrone, medroxyprogesterone, progesterone, and norgestrel.

Typical male sex hormones which may be utilized may be represented by, without limitation, testosterone, methyltestosterone, and fluoxymesterone.

The composition can include the (C) personal care ingredient in various amounts. One of ordinary skill in the art can readily select an appropriate amount based on want or need. Further, one of ordinary skill in the art readily understands how to select at least one of the (C) personal care ingredients for preparing the composition in view of the desired application/function thereof. For example, the relative amounts of the components of the composition are contingent on the presence or absence of various optional components, along with the desired properties of the composition and its end use. One of skill in the art readily understands how to optimize relative amounts of these components.

The composition may further include a filler. Examples of fillers include talc, micas, kaolin, zinc or titanium oxides, calcium or magnesium carbonates, silica, silica silylate, titanium dioxide, glass or ceramic beads, polymethylmethacrylate beads, boron nitride, aluminum silicate, aluminum starch octenylsuccinate, bentonite, magnesium aluminum silicate, nylon, silk powder metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, silicone resin microbeads, and mixtures thereof. The fillers may be surface treated to modify affinity or compatibility with remaining components.

A method of preparing the composition is also disclosed. The method comprises combining the (A) silicone resin-linear polymer; the (B) carrier fluid; and when the composition is the personal care composition, the (C) personal care ingredient.

The components may be combined in any order, optionally under shear or mixing. Parameters associated with reaction conditions may also be controlled, e.g. temperature, pressure, etc. However, the method may be carried out at ambient conditions

In embodiments in which the composition is in the form of a gel paste, the gel paste may have a viscosity of at least 50, alternatively at least 100, or alternatively at least 200, Pa·s, as measured on a Brookfield DVII+viscometer with Helipath attachment using spindle T-D (20.4 mm crossbar) at 2.5 rpm.

In a specific embodiment, the composition is an emulsion. The emulsion can be formed by combining the (A) silicone resin-linear copolymer, or the composition including it, with water, optionally under shear, and optionally in the presence of an emulsifying agent. In certain embodiments, the emulsifying agent is present in the emulsion, and the emulsion is formed with shear. “Shearing”, as used herein refers to any shear mixing process, such as obtained from homogenizing, sonalating, or any other mixing processes known in the art as shear mixing.

Shearing can be accomplished by any method known in the art to affect mixing of high viscosity materials. The mixing may occur either as a batch, semi-continuous, or continuous process. Mixing may occur, for example using, batch mixing equipment with medium/low shear include change-can mixers, double-planetary mixers, conical-screw mixers, ribbon blenders, double-arm or sigma-blade mixers. Illustrative examples of continuous mixers/compounders include extruders single-screw, twin-screw, and multi-screw extruders, corotating extruders, twin-screw counter-rotating extruders, two-stage extruders, twin-rotor continuous mixers, dynamic or static mixers or combinations of these equipment.

The amount of water utilized may vary. In certain embodiments, water forms a continuous phase in the emulsion. In other embodiments, water forms a discontinuous phase in the emulsion.

The emulsifying agent may be selected from any ionic, nonionic, or zwitterionic surfactant capable of stabilizing emulsions. The emulsifying agent may be an anionic surfactant, cationic surfactant, nonionic surfactant, amphoteric surfactant, or a combination thereof.

Representative examples of suitable anionic surfactants include alkali metal soaps of higher fatty acids, alkylaryl sulphonates such as sodium dodecyl benzene sulphonate, long chain fatty alcohol sulphates, olefin sulphates and olefin sulphonates, sulphated monoglycerides, sulphated esters, sulphonated ethoxylated alcohols, sulphosuccinates, alkane sulphonates, phosphate esters, alkyl isethionates, alkyl taurates, and alkyl sarcosinates. Representative examples of suitable cationic surfactants include alkylamine salts, quaternary ammonium salts, sulphonium salts, and phosphonium salts. Representative examples of suitable nonionic surfactants include condensates of ethylene oxide with long chain fatty alcohols or fatty acids such as a C12-16 alcohol, condensates of ethylene oxide with an amine or an amide, condensation products of ethylene and propylene oxide, esters of glycerol, sucrose, sorbitol, fatty acid alkylol amides, sucrose esters, fluoro-surfactants, and fatty amine oxides. Representative examples of suitable amphoteric surfactants include imidazoline compounds, alkylamino acid salts, and betaines.

Treatment methods with the personal care composition are also provided. For example, when the composition is a skin care composition, the method comprises the step of administering the composition to skin of a subject.

The treatment method comprises applying the composition to a substrate. Generally, the substrate comprises a portion of a mammal, particularly a human. One specific example of a suitable substrate is skin. However, the substrate need not be skin or dermis. For example, when the personal care composition comprises the hair care composition, the substrate is typically hair, which is a protein filament that grows from the follicles of skin. Alternatively, when the personal care composition is the nail care composition, the substrate is a nail, which comprises keratin. Alternatively still, when the personal care composition is the tooth care composition, the substrate is at least one tooth.

The step of applying may be carried out via any technique for contacting the substrate with the composition. For example, the composition may simply be applied to the substrate by a user, e.g. the user supplying the substrate, or by another. The composition may be dispensed, spread, and/or applied on the substrate, optionally while applying a force to spread or apply the composition. In certain embodiments, the substrate may also take the form of a bandage or similar article. Such articles can thus carry and deliver the composition to the user's skin when contacted. Alternatively, the bandage or other article may be at least partially coated with the composition, and the substrate is contacted with the composition by applying and optionally adhering the bandage or other article including the composition to the substrate, e.g. the user's skin. As another example, when the personal care composition comprises the tooth care composition, the tooth care composition may contact the substrate (e.g. teeth) by applying via a brush.

More specifically, when the composition comprises the hair care composition, the hair care composition may be used on hair in a conventional manner. An effective amount of the composition for washing or conditioning hair is applied to the hair. Such effective amounts generally range from 1 to 50, alternatively from 1 to 20, grams. Application to the hair typically includes working the composition through the hair such that most or all of the hair is contacted with the composition. These steps can be repeated as many times as desired to achieve the desired benefit.

Benefits obtained from using the hair care composition on hair include one or more of the following benefits: color retention, improvement in coloration process, hair conditioning, softness, detangling ease, silicone deposition, anti-static, anti-frizz, lubricity, shine, strengthening, viscosity, tactile, wet combing, dry combing, straightening, heat protection, styling, or curl retention.

When the composition comprises the skin care composition, the skin care composition may be used on skin in a conventional manner. An effective amount of the composition for the purpose is applied to the skin. Such effective amounts generally range from 1 to 3, mg/cm². Application to the skin typically includes working the composition onto or into the skin. This method for applying to the skin comprises the steps of contacting the skin with the composition in an effective amount and then rubbing the composition into the skin. These steps can be repeated as many times as desired to achieve the desired benefit.

Benefits obtained from using the skin care composition on skin include one or more of the following benefits: stability in various formulations (o/w, w/o, anhydrous), utility as an emulsifier, level of hydrophobicity, organic compatibility, substantivity/durability, wash off resistance, interactions with sebum, performance with pigments, pH stability, skin softness, suppleness, moisturization, skin feel, long lasting, long wear, long lasting color uniformity, color enhancement, foam generation, optical effects (soft focus), stabilization of actives.

Methods of preparing the silicone resin-linear copolymer are also disclosed.

In a first embodiment, the method comprises reacting a linear organopolysiloxane having at least one silicon-bonded hydroxyl group and an acetoxysilane to give an acetoxysilylated organopolysiloxane. The silicon-bonded hydroxyl group is typically terminal in the linear organopolysiloxane. However, additional silicon-bonded hydroxyl groups may be present in pendent locations.

The linear organopolysiloxane becomes the (A2) linear structure of the silicone resin-linear copolymer. The linear organopolysiloxane may be selected based on desired characteristics, including DP, of the silicone resin-linear copolymer. The acetoxysilane typically has the formula R²_{q}SiX_{4-q}, where R² is a substituted or unsubstituted hydrocarbyl group; X is an acetoxy group, and q is an integer selected from 0 or 1 to 3. In certain embodiments, q is 1 such that the acetoxysilane is a trialkylacetoxysilane. Examples of trialkylacetoxysilanes include methyltriacetoxysilane, ethyltriacetoxysilane, or a combination of both. The acetoxy groups of the above formula may be replaced with any hydrolysable group in alternative methods.

The reaction between the linear organopolysiloxane having at least one silicon-bonded hydrogen atom and the acetoxysilane is a hydrolysis/condensation reaction. In various embodiments, the reaction is carried out in the presence of a catalyst, which can be any condensation catalyst. The reaction can be carried out in a solvent, e.g. any of the suitable carrier fluids described above.

Examples of suitable condensation catalyst include acids, such as carboxylic acids, e.g. formic acid, acetic acid, propionic acid, butyric acid, and/or valeric acid; bases; metal salts of organic acids, such as dibutyl tin dioctoate, iron stearate, and/or lead octoate; titanate esters, such as tetraisopropyl titanate and/or tetrabutyl titanate; chelate compounds, such as acetylacetonato titanium; transition metal catalysts, such as platinum-containing catalysts, including for example any of those introduced above as being suitable hydrosilylation catalysts; aminopropyltriethoxysilane, and the like. If utilized, the condensation catalyst are typically utilized in a catalytic amount, e.g. in amount of from greater than 0 to 5, alternatively 0.0001 to 1, alternatively 0.001 to 0.1, percent by weight, based on 100 parts by weight based on the combined weight of the linear organopolysiloxane and the acetoxysilane.

The relative amounts of the acetoxysilane and the linear organopolysiloxane may vary. In a specific embodiment, the relative amount of acetoxysilane is calculated based on a ratio of mols silicon from the acetoxysilane to mols silicon from the silicone resin×100, with the ratio typically being from greater than 0 to 12, alternatively from 1 to 10, alternatively from 1 to 8.

The acetoxysilylated organopolysiloxane is linear and typically includes the acetoxysilyl group in a terminal location. In this first embodiment, the method further comprises reacting the acetoxysilylated organopolysiloxane with a silicone resin having at least one silicon-bonded hydroxyl group to give the silicone resin-linear copolymer. The reaction can be carried out in a solvent, e.g. any of the suitable carrier fluids described above.

The silicone resin typically has the formula R¹SiO_{3/2}, which corresponds to the (A1) resinous structure of the silicone resin-linear copolymer. The silicone resin typically includes at least some silicon-bonded hydroxyl groups, as understood in the art. As such, the acetoxysilylated organopolysiloxane and the silicone resin also react via a hydrolysis/condensation reaction, which may be catalyzed as set forth above. The silicone resin may have a molecular weight selected based on desired characteristics of the silicone resin-linear copolymer.

The relative amounts of the acetoxysilylated organopolysiloxane and the silicone resin may vary and are typically selected based on the desired ratio or amounts of the (A1) resinous structure and (A2) linear structure in the silicone resin-linear copolymer. The silicone resin is topically utilized in an amount of from 10 to 70 wt. %, alternatively from 20 to 55 wt. %, alternatively from 30 to 50 wt. %, alternatively from 40 to 50 wt. %, based on the combined weight of the acetoxysilylated organopolysiloxane and the silicone resin.

In a second embodiment, the method comprises reacting a linear organopolysiloxane having at least one silicon-bonded hydrogen atom, hydroxyl group, or alkoxy group and a silicone resin having at least one silicon-bonded hydrogen atom, hydroxyl group, or alkoxy group in the presence of a Lewis acid catalyst. This reaction is a dehydrogenation reaction.

When the linear organopolysiloxane includes the silicon-bonded hydrogen atom, the silicone resin includes a silicon-bonded hydroxyl group or alkoxy group, and when the linear organopolysiloxane includes the silicon-bonded hydroxyl group or alkoxy group, the silicone resin includes the silicon-bonded hydrogen atom.

The silicon-bonded hydrogen atom, hydroxyl group, or alkoxy group of the linear organopolysiloxane is typically terminal in the linear organopolysiloxane. However, additional silicon-bonded hydrogen atoms, hydroxyl groups, or alkoxy groups may be present in pendent locations.

The dehydrogenation reaction can be carried out in a solvent, e.g. any of the suitable carrier fluids described above. Typically, the solvent, the linear organopolysiloxane, and the silicone resin are disposed in a vessel and heated to reflux. Typically, the contents of the vessel become a homogenous solution. The Lew acid catalyst is then disposed in the vessel, which results in the formation of gaseous byproducts (hydrogen, methane). The amount of Lewis acid catalyst may vary, e.g. from greater than 0 to 1,000, alternatively from 50 to 500, alternatively from 100 to 200, parts per million (ppm) based on total solids content.

The Lewis acid catalyst may be any Lewis acid catalyst suitable for the dehydrogenation reaction. Suitable Lewis acid catalysts for use herein include alumoxane (modified and unmodified), C1-30 hydrocarbyl substituted Group 13 compounds, including, for example, tri(hydrocarbyl)aluminum- or tri(hydrocarbyl)boron compounds and halogenated (including perhalogenated) derivatives thereof, having from 1 to 10 carbons in each hydrocarbyl or halogenated hydrocarbyl group. In specific embodiments, the Lewis acid catalyst comprises a perfluorinated tri(aryl)boron compound, e.g. tris(pentafluorophenyl)borane.

The Lewis acid catalyst may be removed via a neutral powder and filtration, e.g. alumina powder.

Embodiment 1 relates to a silicone resin-linear co-polymer comprising a resinous structure (A1) including R¹SiO_{3/2} units, and a linear structure (A2) including repeated R²₂SiO_{2/2} units, wherein R¹ is a propyl group and each R² is an independently selected substituted or unsubstituted hydrocarbyl group, and wherein the resinous structure (A1) and the linear structure (A2) are bonded together in the silicone resin-linear copolymer via a siloxane bond.

Embodiment 2 relates to the silicone resin-linear copolymer of Embodiment 1 consisting of the resinous structure (A1) and the linear structure (A2).

Embodiment 3 relates to the silicone resin-linear copolymer of Embodiment 1 or 2 wherein the resinous structure (A1) consists of R¹SiO_{3/2} units, wherein R¹ is defined above.

Embodiment 4 relates to the silicone resin-linear copolymer of any one of Embodiments 1-3 claim wherein the linear structure (A2) consists of R²₂SiO_{2/2} units, wherein R² is defined above.

Embodiment 5 relates to the silicone resin-linear copolymer of any one of Embodiments 1-4, wherein the siloxane bond between the resinous structure and the linear structure is derived from an acetoxysilyl group.

Embodiment 6 relates to the silicone resin-linear copolymer of any one of Embodiments 1-4, wherein the siloxane bond between the resinous structure and the linear structure is derived dehydrogenation with a Lewis acid catalyst.

Embodiment 7 relates to a composition, comprising: (A) the silicone resin-linear co-polymer of any one of Embodiments 1-6; and (B) a carrier fluid.

Embodiment 8 relates to the composition of Embodiment 7, wherein the (B) carrier fluid comprises (B1) a volatile fluid at 25° C.

Embodiment 9 relates to the composition of Embodiment 7 or 8 further defined as a cosmetic composition.

Embodiment 10 relates to a film-forming agent comprising the silicone resin-linear co-polymer of any one of Embodiments 1-6.

Embodiment 11 relates to an adhesion promotor comprising the silicone resin-linear co-polymer of any one of Embodiments 1-6

Embodiment 12 relates to an encapsulant comprising the silicone resin-linear co-polymer of any one of Embodiments 1-6.

Embodiment 13 relates to an electric device including a film formed from the silicone resin-linear co-polymer of any one of Embodiments 1-6.

Embodiment 14 relates to a method of preparing a conformal coating on an electronic device, the method comprising:

applying a composition on the electronic device; and

forming the conformal coating on the electronic device;

wherein the composition is the composition of Embodiment 7 or 8.

Embodiment 16 relates to a method of preparing a silicone resin-linear copolymer, the method comprising:

reacting a linear organopolysiloxane having at least one silicon-bonded hydroxyl group and an acetoxysilane to give an acetoxysilylated organopolysiloxane; and

reacting the acetoxysilylated organopolysiloxane with a silicone resin having at least one silicon-bonded hydroxyl group to give the silicone resin-linear copolymer;

wherein the silicone resin-linear copolymer is that of any one of Embodiments 1-6.

Embodiment 16 relates to a method of preparing a silicone resin-linear copolymer, the method comprising:

reacting a linear organopolysiloxane having at least one silicon-bonded hydrogen atom, hydroxyl group, or alkoxy group and a silicone resin having at least one silicon-bonded hydrogen atom, hydroxyl group, or alkoxy group in the presence of a Lewis acid catalyst;

with the proviso that when the linear organopolysiloxane includes the silicon-bonded hydrogen atom, the silicone resin includes a silicon-bonded hydroxyl group or alkoxy group, and when the linear organopolysiloxane includes the silicon-bonded hydroxyl group or alkoxy group, the silicone resin includes the silicon-bonded hydrogen atom; and

wherein the silicone resin-linear copolymer is that of any one of Embodiments 1-6.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The following examples are intended to illustrate the invention and are not to be viewed in any way as limiting to the scope of the invention.

Silicone resin-linear copolymers are prepared in accordance with the present invention.

### PREPARATION EXAMPLE 1

800 grams of deionized water are disposed in a flask equipped with a stir paddle, thermometer, and a water-cooled condenser. Water is cooled to 5° C. via an ice-water bath. A pre-mixed solution of propyltrichlorosilane (304 g) in toluene (200 g) is disposed in the flask over 1-2 minutes at a mixing speed of 400 rpm. The ice-water bath is removed with continued mixing for 5 minutes. Maximum temperature reached in the flask is 74° C. During the 5 minute time period of continued mixing the temperature drops to 70° C. The contents of the flask are transferred reaction mixture into a 1 L 3-neck round bottom flask with a bottom drain. The aqueous layer is removed. HCl is removed by repeatedly adding 37.0 grams of deionized water, heating for three minutes, and further removal of the aqueous layer. This process is repeated until the final contents of the flask had a pH of 4-4.5. 2-propanol (9.3 g) and deionized water (27.7 g) are disposed in the flask, and the flask is heated at 80° C. for a few more minutes. The aqueous phase is again removed. The contents of the flask are heated to reflux to remove residual water by azeotropic distillation. After mixing for several minutes at 80° C. and removal of the aqueous phase, the pH is ˜4. The contents of the flask are again heated to reflux and any aqueous phase is again removed. A reaction mixture comprising a silicone resin results, which is pressure filtered through an Osmonics MAGNA Nylon Supported Plain 5.0 um filter (47 mm diameter). The reaction product is optically clear and colorless. The silicone resin (T-propyl) has a Mn of 2,590 and a Mw of 6,050 (using Gel Permeation Chromatography (GPC) techniques based on polystyrene standards). The silicone resin had 58.8 mol % OH as measured via NMR.

### EXAMPLE 1

56.98 grams of a solution comprising the silicone resin of Preparation Example 1 (45.0 grams solids in toluene) and 55.0 grams of Si—H terminated polydimethylsiloxane (DP of 184) are disposed in a flask. The flask is equipped with a thermometer, stir paddle, and a Dean Stark apparatus attached to a water-cooled condenser. A nitrogen blanket is applied. A heating mantle is used for heating. The Dean Stark was prefilled with toluene. The amount of toluene added results in a solids content of 52.9 wt. %. The contents of the flask are heated at reflux for 30 minutes to remove residual water and cooled to 85° C. At 85° C., 0.40 grams of a Lewis Acid catalyst (B(C₆F₅)₃) in solution to provide 200 ppm based on solids is disposed in the flask, and the contents of the flask are heated at 85° C. for 30 min. Some toluene is distilled off to provide a NVC of ˜60%. The contents of the flask are heated at reflux for 3 hours, followed by the addition of tetramethyldisiloxane. After cooling to ˜28° C., 1.20 grams of tetramethyldisiloxane are disposed in the flask. The contents of the flask are mixed while cooling to room temperature for 30 minutes. At 50° C., 0.40 grams of a Lewis Acid catalyst (B(C₆F₅)₃) in solution to provide 200 ppm based on solids is disposed in the flask, and the flask is heated at 50° C. for 30 minutes. The next day, neutral aluminum oxide (2.0 grams) are disposed in the flask and mixed for several hours at room temperature. A reaction mixture comprising a silicone resin-linear copolymer results. The reaction mixture is pressure filtered through a 142 mm diameter Magna, Nylon, Supported, Plain, 5.0 Micron filter. The silicone resin-linear copolymer has a Mn of 42,000 and a Mw of 121,000 g/mole (using Gel Permeation Chromatography (GPC) techniques based on polystyrene standards). The silicone resin-linear copolymer had 19.7 mol % OH as measured via NMR. The silicone resin-linear copolymer forms films that are optically transparent, which is desirable.

### PROPHETIC EXAMPLE 1

48.89 grams of polydimethylsiloxane (PDMS) having a DP of 184 and a silicon-bonded hydroxyl group at each terminal in toluene (65 wt. % of the PDMS in toluene) and 1.59 grams of an acetoxysilane (50/50 molar ratio of methyltriacetoxysilane and ethyltriacetoxysilane) are disposed in a flask and allowed to react for one hour at room temperature to give an acetoxysilylated PDMS. 40 grams of a silicone resin (T^{Pr}, 42 wt. % in toluene) having at least one silicon-bonded hydroxyl group is disposed in a flask to give a weight ratio of the acetoxysilylated PDMS to the silicone resin of 45:55. The reaction is carried out at 90° C. for 4 hours to give a silicone resin-linear copolymer. Non-reactive volatile solids content was maintained at 28% to maintain an optically clear solution during the reaction.

### PROPHETIC EXAMPLE 2

55 grams of polydimethylsiloxane (PDMS) having a DP of 184 and a silicon-bonded hydroxyl group at each terminal in toluene (30.1 wt. % of the PDMS in toluene) and 45 grams solids of a silicone resin (T^{Pr} 50.95 wt. % in toluene) having at least one silicon-bonded hydroxyl group is disposed in a flask and heated to reflux for 30 minutes. At 85° C. 0.4 grams of a Lewis acid catalyst (tris-pentafluoroborane in toluene to provide 200 ppm of tris-pentafluoroborane) is disposed in the flask. Bubbling and foaming occurs, evidencing the formation of gas and initiation of a dehydrogenation reaction. The contents of the flask are heated at reflux for 30 minutes to one hour to give a reaction product including a silicone resin-linear copolymer. The Lewis acid catalyst is removed from the reaction product by adsorption onto alumina powder and subsequent filtration. Alumina powder was utilized in an amount of 2 wt. % based on solids. The dehydrogenation reaction obviates any undesirable byproducts from the reaction product.

The invention has been described in an illustrative manner, and it is to be understood that the terminology which has been used is intended to be in the nature of words of description rather than of limitation. Obviously, many modifications and variations of the present invention are possible in light of the above teachings. The invention may be practiced otherwise than as specifically described.

1. A silicone resin-linear co-polymer comprising a resinous structure (A1) including R¹SiO_{3/2} units, and a linear structure (A2) including repeated R²₂SiO_{2/2} units, wherein R¹ is a propyl group and each R² is an independently selected substituted or unsubstituted hydrocarbyl group, and wherein the resinous structure (A1) and the linear structure (A2) are bonded together in the silicone resin-linear copolymer via a siloxane bond.

2. The silicone resin-linear copolymer of claim 1 consisting of the resinous structure (A1) and the linear structure (A2).

3. The silicone resin-linear copolymer of claim 1, wherein the resinous structure (A1) consists of R¹SiO_{3/2} units where R¹ is as defined above.

4. The silicone resin-linear copolymer of claim 1, wherein the linear structure (A2) consists of R²₂SiO_{2/2} units where R² is as defined above.

5. The silicone resin-linear copolymer of claim 1, wherein the siloxane bond between the resinous structure (A1) and the linear structure (A2) is derived from an acetoxysilyl group.

6. The silicone resin-linear copolymer of claim 1, wherein the siloxane bond between the resinous structure (A1) and the linear structure (A2) is derived from dehydrogenation with a Lewis acid catalyst.

7. A composition comprising:
(A) the silicone resin-linear co-polymer of claim 1; and
(B) a carrier fluid.

8. The composition of claim 7, wherein:
(i) the (B) carrier fluid comprises (B1) a volatile fluid at 25° C.;
(ii) the composition is further defined as a cosmetic composition; or
(iii) both (i) and (ii).

9. A film-forming agent comprising the silicone resin-linear co-polymer of claim 1.

10. An adhesion promotor comprising the silicone resin-linear co-polymer of claim 1.

11. An encapsulant comprising the silicone resin-linear co-polymer of claim 1.

12. An electric device including a film formed from the silicone resin-linear co-polymer of claim 1.

13. A method of preparing a conformal coating on an electronic device, said method comprising:
applying a composition on the electronic device; and
forming the conformal coating on the electronic device;
wherein the composition is the composition of claim 7.

14. A method of preparing a silicone resin-linear copolymer, said method comprising:
reacting a linear organopolysiloxane having at least one silicon-bonded hydroxyl group and an acetoxysilane to give an acetoxysilylated organopolysiloxane; and
reacting the acetoxysilylated organopolysiloxane with a silicone resin having at least one silicon-bonded hydroxyl group to give the silicone resin-linear copolymer;
wherein the silicone resin-linear copolymer is that of claim 1.

15. A method of preparing a silicone resin-linear copolymer, said method comprising:
reacting a linear organopolysiloxane having at least one silicon-bonded hydrogen atom, hydroxyl group, or alkoxy group and a silicone resin having at least one silicon-bonded hydrogen atom, hydroxyl group, or alkoxy group in the presence of a Lewis acid catalyst;
with the proviso that when the linear organopolysiloxane includes the silicon-bonded hydrogen atom, the silicone resin includes a silicon-bonded hydroxyl group or alkoxy group, and when the linear organopolysiloxane includes the silicon-bonded hydroxyl group or alkoxy group, the silicone resin includes the silicon-bonded hydrogen atom; and
wherein the silicone resin-linear copolymer is that of claim 1.
